(19) United States
(12) Patent Application Publication
(10) Pub. No.: US 2008/0261305 A1
(43) Pub. Date: Oct. 23, 2008

(54) TGF-beta-MEDIATED OSTEOGENIC DIFFERENTIATION OF MESENCHYMAL STEM CELLS

(76) Inventors: Basil M. Hantash, E. Palo Alto, CA (US); Longmei Zhao, Mountain View, CA (US)

Correspondence Address: Bozicevic, Field & Francis LLP;Stanford University Office of Technology Licensing, 1900 University Avenue, Suite 200, East Palo Alto, CA, 94303, US

(21) Appl. No.: 12/050,034

(22) Filed: Mar. 17, 2008

Related U.S. Application Data
(60) Provisional application No. 60/919,349, filed on Mar. 21, 2007.

(51) Int. Cl. C12N 5/02

(52) U.S. Cl. 435/378

(57) ABSTRACT

Methods and compositions are provided for the culture of MSC to provide osteogenic progenitor cells.

### INTRODUCTION

Multipotent stem cell populations found in adult tissues have been of great interest because they serve as reservoirs for tissue renewal after trauma, disease, and aging. One important type of adult stem cell, known as the mesenchymal stem cell (MSC), is derived from bone marrow. MSCs are maintained in a relative state of quiescence in vivo, but in response to a variety of physiological and pathological stimuli, are capable of proliferating then differentiating into osteoblasts, chondrocytes, adipocytes, or hematopoiesis-supporting stromal cells. However, little is understood regarding the cellular or molecular events underlying MSC fate decisions.

Transforming growth factor β (TGF-β) proteins play important roles in the regulation of many developmental processes and maintenance of normal tissue homeostasis (Massague, J. (1998). Annu Rev Biochem 67: 753-91). TGF-β initiates signaling by binding and activating the membrane-anchored type II and type I receptor Serine/Threonine kinases, which subsequently phosphorylate the effectors Smad2/Smad3. Phosphorylated Smad2/Smad3 can then form complexes with Smad4 and translocate into the nucleus. Accumulation of Smads in the nucleus results in the activation or repression of downstream target genes by recruiting various transcriptional coactivators or corepressors (Derynck, R. (1998) Nature 393(6687): 737-9). The central regulation of bone differentiation and formation is controlled by the transcriptional activity of Runx2 and TAZ. Essential during osteogenic differentiation of mesenchymal progenitor cells, homozygous deletion of Runx2 in mice results in the complete absence of osteoblasts and bone (Komori et al. (1997) Cell 89(5): 755-64; Otto et al. (1997) Cell 89(5):765-71). TAZ, a WW domain-containing molecule, functions as a transcriptional modulator to stimulate bone development while simultaneously blocking the differentiation of mesenchymal stem cells into fat. These developmental effects occur through direct interaction between TAZ and the transcription factors Runx2 and PPARγ, resulting in transcriptional enhancement and repression, respectively, of selective programs of gene expression (Hong (2006) Cell Cycle 5(2):176-9; Hong et al. (2005) Science 309(5737):1074-8.

The transforming growth factor-β (TGF-β) pathway plays a key role in the balance between stem cell renewal and differentiation and has been implicated in chondrogenesis. However, the role of TGF-β in osteogenic differentiation remains unclear. In the present study, we investigated the role of TGF-β1 in multipotent differentiation of mouse bone marrow-derived MSCs (BMSCs) in vitro.

### SUMMARY OF THE INVENTION

Compositions and methods are provided that relate to cultures for the osteogenic differentiation of mesenchymal stem cells. MSC are cultured in the presence of a TGB-β, including TGF-β1, or TGF-β3, in an amount sufficient to induce osteogenic differentiation. The osteogenic progenitor cells thus derived may be isolated from the culture, or otherwise utilized for various purposes. For example, osteogenic cells of the invention find use in therapeutic methods, e.g. for transplantation as a source of osteogenic progenitors; and the like.

### BRIEF DESCRIPTION OF THE FIGURES

FIG. 1. Isolation of bone marrow-derived MSCs and detection of Sca-1 in MSCs (A) Mesenchymal stem cells at passage 5 isolated from adult mouse femur bone marrow were attached to tissue culture plate 24 hours after seeding. (B) Nuclear staining using DAPI for all bone marrow-derived MSCs in the field. (C) Staining of same cells shown in B with anti-Sca-1 antibody. Staining is consistent with membrane pattern, the expected location of Sca-1.

FIG. 2. Osteogenic and adipogenic differentiation of MSC. (A) and (C) MSCs were cultured in regular maintain medium for 7 day. MSCs were cultured in osteogenic medium (B) and adipogenic medium (D) for 7 days. Alizarin red staining was performed on (A) and (B). Red Oil Staining was performed on (C) and (D).

FIG. 3. TGF-β1 enhanced expression of osteogenic genes and inhibited expression of adipogenic genes. Bone marrow-derived MSCs treated with TGF-β1 at 10 ng/ml for 2 weeks. RNA was collected from cells and real-time PCR was used to exam the expression of stem cells markers (A), osteogenic genes (B), and adipogenic genes (C).

FIG. 4. Alkaline phosphatase activity at 2 weeks significantly increased in bone marrow-derived MSCs treated with TGF-β1 at 10 ng/ml compared with untreated group. Alkaline Phosphatase (AP) Staining on MSCs cultured for 2 weeks with (B) or without (A) 10 ng/ml of TGF-β1. (C) Alkaline phosphatase activity was measured on MSCs treated with TGF-β1 at 10 ng/ml for 2 weeks.

FIG. 5. The expression of TGF-β1 signaling pathway on bone marrow-derived MSCs. MSCs treated with TGF-β1 at 10 ng/ml for 2 weeks. RNA was collected from cells and real-time PCR was used to exam the expression of TGF-β1, Smad-2, and Smad-3.

### DETAILED DESCRIPTION OF THE EMBODIMENTS

Compositions and methods are provided that relate to cultures for the osteogenic differentiation of mesenchymal stem cells. MSC, which may be bone marrow derived (BMSC) or adipose tissue derived (AMSC) are cultured in the presence of TGB-β, including TGF-β1, or TGF-β3, in an amount sufficient to induce osteogenic differentiation, usually for at least one week and not more than about 3 weeks. The resulting osteoblasts may be implanted into injured bone to promote fracture healing. TGF-β1 and/or -β3 may also be injected around the wound or intravenously to enhance mesenchymal progenitors at the repair site to differentiate into osteoblasts, in order to accelerate the healing process. MSCs will move to the site of injury using homing signals and contribute to bone regeneration at the site of bone injury.

There are two types of bone ossification, intramembranous and endochondral. The former gives rise to flat bones, especially skull and clavicle. Intramembranous ossification does not go through a step of laying down provisional cartilage. MSCs may first convert into osteochondral progenitors by downregulating adipogenic pathway through TAZ upregulation via TGF-β1 or TGF-β3. Then, the specific hormone may promote either intramembranous or endochondral differentiation. This cell dual capability is evidenced in FIG. 6, which shows that exposure to TGF-β1 or TGF-β3 resulted in osteoblast matrix deposition, as evidenced by positive alkaline phosphatase activity using cultured cells. In particular, TGF-β1 down-regulates chondrogenesis and increases osteogenesis.

Advantages of the present invention include the use of a biologic agent, human recombinant TGF-β, which can serve as a therapeutic agent for bone healing via osteogenic stimulation. This simple treatment is physiological, unlike the artificial treatments such as dexamethasone, β-glycerophosphate and ascorbic acid that are currently employed for bone engineering. For example, the use of dexamethasone is problematic due to the fact that steroids are known inhibitors of wound healing in vivo.

The effect of TGF-β1 in fracture healing may be more effective than other osteogenic cytokines, such as BMP-2 and BMP-4. TGF-β1 in peripheral blood dramatically increases at two weeks after trauma and continue at the high level at six months after trauma, while BMP-2 and BMP-4 in peripheral blood can not be detected during the whole healing process. Therefore, TGF-β offers a physiologically relevant stimulation to bone regeneration, unlike BMPs.

In other embodiments of the invention, adenoviral gene transfer is utilized as a safe and effective way to obtain high and stable expression of protein. A recombinant adenovirus expressing one or both of TGF-β1 and TGF-β3 may be generated and transferred into MSCs to stimulate osteogenesis in vitro.

In other embodiments, a biomaterials scaffold serves as a synthetic extracellular matrix (ECM) to organize cells into a three-dimensional (3D) architecture and to present stimuli, which direct the growth and formation of a desired tissue. Human MSCs are seeded on the desired scaffolds and cultured with human recombination TGF-β1 or TGF-β3 or both to generate osteoblasts in vitro. Scaffolds can be impregnated with recombinant TGF-β1 or TGF-β3 so as to release growth factor over a sustained period of time, e.g. for at least about 3 month 3 and up to about 6 months stimulation of osteogenesis. For example, MSCs can be seeded onto TGF-β1 or TGF-β3 impregnated scaffolds and implanted into a site of bone injury. In some embodiments the seeded MSC are transfected with adenovirus directing expression of one or both of TGF-β1 and TGF-β3.

As an alternative method to in vitro culture, short sequence synthetic peptides of TGF-β1 or TGF-β3, which contain the receptor binding motif, can be generated introduced into the region of a bone fracture, in order to induce endogenous MSCs around the wound to differentiate into osteoblasts.

### DEFINITIONS

Terms used in the claims and specification are defined as set forth below unless otherwise specified.

The term “ameliorating” refers to any therapeutically beneficial result in the treatment of a disease state, e.g., an autoimmune disease state, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to alter a protein expression profile.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an,” and “the” include plural referents unless the context clearly dictates otherwise.

Mesenchymal Stem Cell (MSC). As used herein, the term MSC refers to a cell capable of giving rise to differentiated cells in multiple mesenchymal lineages, specifically to osteoblasts, adipocytes, myoblasts and chondroblasts. Generally, mesenchymal stem cells also have one or more of the following properties: an ability to undergo asynchronous, or symmetric replication, that is where the two daughter cells after division can have different phenotypes; extensive self-renewal capacity; and clonal regeneration of the tissue in which they exist, for example, the non-hematopoietic cells of bone marrow. “Progenitor cells” differ from stem cells in that they typically do not have the extensive self-renewal capacity. In contrast to previously reported MSC and multipotent mesenchymal cell populations, the cells of the invention do not require lengthy time in culture prior to the appearance of the MSC phenotype, i.e. cells with the MSC phenotype and are responsive to canonical wnt signaling pathways are present in freshly isolated or primary cultures that have been cultured for less than about 20 passages; usually less than about 10 passages.

MSC have been harvested from the supportive stroma of a variety of tissues. In both mouse and human a candidate population of cells has been identified in subcutaneous adipose tissue (AMSC). These cells have demonstrated the same in vitro differentiation capacity as BM-MSC for the mesenchymal lineages, osteoblasts, chondrocytes, myocytes, neurons, and adipocytes (Zuk et al. (2002) Mol Biol Cell 13, 4279-95; Fujimura et al. (2005) Biochem Biophys Res Commun 333, 116-21). Additionally, cell surface antigen profiling of these cells has revealed similar cell surface marker characteristics as the more widely studied BM-MSC (Simmons et al. (1994) Prog Clin Biol Res 389, 271-80; and Gronthos et al. (2001) J Cell Physiol 189, 54-63).

MSC may be characterized by both the presence of markers associated with specific epitopes identified by antibodies and the absence of certain markers as identified by the lack of binding of specific antibodies. MSC may also be identified by functional assays both in vitro and in vivo, particularly assays relating to the ability of stem cells to give rise to multiple differentiated progeny; assays for responsiveness to canonical wnt signaling; and the like.

The cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

The cells which are employed may be fresh, frozen, or have been subject to prior culture. They may be fetal, neonate, adult. MSC may be obtained from adipose tissue (see U.S. Patent application 20030082152); bone marrow (Pittenger et al. (1999) Science 284(5411):143-147; Liechty et al. (2000) Nature Medicine 6:1282-1286); G-CSF or GM-CSF mobilized peripheral blood (Tondreau et al. (2005) Stem Cells 23(8): 1105-1112), or any other conventional source.

In some embodiments, the homogeneous MSC composition is stable in non-differentiating culture conditions, where the proportion of cells in the composition that have an MSC phenotype are maintained over multiple passages. Such cells may be maintained for at least about two passages; at least about five passages; at least about ten passages; or more.

Osteogenic culture conditions. Differentiating cells are obtained by culturing or differentiating MSC in a growth environment that enriches for cells with the desired phenotype, e.g. osteoblasts, osteogenic progenitor cells, etc. The culture may comprise agents that enhance differentiation to a specific lineage.

Osteogenic differentiation may be performed by plating cells and culturing to confluency, then culturing in medium comprising TGF-β1 and/or TGF-β3 at a concentration of from around about 100 pg/ml to around about 100 ng/ml, usually around about 10 ng/ml.

Following the differentiation in culture, the culture will usually comprise at least about 25% of the desired differentiated cells; more usually at least about 50% differentiated cells cells; at least about 75% differentiated cells, or more. The cells thus obtained may be used directly, or may be further isolated, e.g. in a negative selection to remove MSCs and other undifferentiated cells. Further enrichment for the desired cell type may be obtained by selection for markers characteristic of the cells, e.g. by flow cytometry, magnetic bead separation, panning, etc., as known in the art.

Analysis or separation by cell staining may use conventional methods, as known in the art. Techniques providing accurate enumeration include confocal microscopy, fluorescence microscopy, fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide).

The affinity reagents may be specific receptors or ligands for the cell surface molecules indicated above. In addition to antibody reagents, polynucleotide probes specific for an mRNA of interest, peptide-MHC antigen and T cell receptor pairs may be used; peptide ligands and receptor; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to cells, and incubated for a period of time sufficient to bind the available antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The cells of interest may be separated from a complex mixture of cells by techniques that enrich for cells having the above described characteristics. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions highly enriched for osteogenic progenitors are achieved in this manner. The subject population may be at or about 50% or more of the cell composition, and preferably be at or about 75% or more of the cell composition, and may be 90% or more. The desired cells are identified by their surface phenotype, by the ability to develop into bone, etc. The enriched cell population may be used immediately, or may be frozen at liquid nitrogen temperatures and stored for long periods of time, being thawed and capable of being reused. The cells will usually be stored in 10% DMSO, 50% FCS, 40% RPMI 1640 medium.

In one embodiment of the invention, a nucleic acid construct is introduced into the cells of the invention. A variety of vectors are known in the art for the delivery of sequences into a cell, including plasmid vectors, viral vectors, and the like. In a preferred embodiment, the vector is a retroviral or lentiviral vector. For example, see Baum et al. (1996) J Hematother 5(4):323-9; Schwarzenberger et al. (1996) Blood 87:472-478; Nolta et al. (1996) P.N.A.S. 93:2414-2419; and Maze et al. (1996) P.N.A.S. 93:206-210, Mochizuki et al. (1998) J Virol 72(11):8873-83. The use of adenovirus based vectors with hematopoietic cells has also been published, see Ogniben and Haas (1998) Recent Results Cancer Res 144:86-92.

Various techniques known in the art may be used to transfect the target cells, e.g. electroporation, calcium precipitated DNA, fusion, transfection, lipofection and the like. The particular manner in which the DNA is introduced is not critical to the practice of the invention.

In some embodiments of the invention, a pharmaceutical composition of the present invention is administered to an animal to accelerate bone repair, e.g. following an injury, in the treatment of bone disease, etc.

A cell transplant, as used herein, is the transplantation of one or more cells into a recipient body, usually for the purpose of augmenting function of an organ or tissue in the recipient. As used herein, a recipient is an individual to whom tissue or cells from another individual (donor), commonly of the same species, has been transferred. The graft recipient and donor are generally mammals, preferably human. Laboratory animals, such as rodents, e.g. mice, rats, etc. are of interest for drug screening, elucidation of developmental pathways, etc. For the purposes of the invention, the cells may be allogeneic, autologous, or xenogeneic with respect to the recipient.

Where the transplantation is intended for the treatment of degenerative disease, e.g. osteogenesis imperfecta; repair of mesenchymal tissues; etc., the cells are administered in a manner that permits them to graft or migrate to the intended tissue site and reconstitute or regenerate the functionally deficient area.

In many clinical situations, the bone healing condition are less ideal due to decreased activity of bone forming cells, e.g. within aged people, following injury, in osteogenesis imperfecta, etc. A variety of bone and cartilage disorders affect aged individuals. Such tissues are normally regenerated by mesenchymal stem cells. Included in such conditions is osteoarthritis. Osteoarthritis occurs in the joints of the body as an expression of “wear-and-tear”. Thus athletes or overweight individuals develop osteoarthritis in large joints (knees, shoulders, hips) due to loss or damage of cartilage. This hard, smooth cushion that covers the bony joint surfaces is composed primarily of collagen, the structural protein in the body, which forms a mesh to give support and flexibility to the joint. When cartilage is damaged and lost, the bone surfaces undergo abnormal changes. There is some inflammation, but not as much as is seen with other types of arthritis. Nevertheless, osteoarthritis is responsible for considerable pain and disability in older persons.

In conditions of the aged where repair of mesenchymal tissues is decreased, or there is a large injury to mesenchymal tissues, the osteogenic activity may be enhanced by administration of progenitor cells.

In methods of accelerating bone repair, a pharmaceutical composition of the present invention is administered to a patient suffering from damage to a bone, e.g. following an injury. The formulation is preferably administered at or near the site of injury, following damage requiring bone regeneration.

In the methods of the invention, cells to be transplanted are transferred to a recipient in any physiologically acceptable excipient comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996. Choice of the cellular excipient and any accompanying elements of the composition will be adapted in accordance with the route and device used for administration. The cells may be introduced by injection, catheter, or the like. The cells may be frozen at liquid nitrogen temperatures and stored for long periods of time, being capable of use on thawing. If frozen, the cells may be stored in a 10% DMSO, 50% FCS, 40% RPMI 1640 medium.

### Expression Assays

The in vitro differentiated cells find use in the examination of gene expression. The expressed set of genes may be compared with a variety of cells of interest, e.g. adult osteoblasts and progenitors thereof, etc, as known in the art. For example, one could perform experiments to determine the genes that are regulated during development.

Any suitable qualitative or quantitative methods known in the art for detecting specific mRNAs can be used. mRNA can be detected by, for example, hybridization to a microarray, in situ, hybridization in tissue sections, by reverse transcriptase-PCR; or in Northern, blots containing-poly⁺ mRNA. One of skill in the art can readily use these methods to determine differences in the size or amount of mRNA transcripts between two samples. For example, the level of particular mRNAs in progenitor cells is compared with the expression of the mRNAs in a reference sample, e.g. hepatocytes, or other differentiated cells.

Hybridization to arrays may be performed, where the arrays can be produced according to any suitable methods known in the art. For example, methods of producing large arrays of oligonucleotides are described in U.S. Pat. Nos. 5,134,854, and 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with arrays are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

In other screening methods, the test sample is assayed at the protein level. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods of qualitative or quantitative detection of levels or amounts of differentially expressed polypeptide can be used, for example ELISA, western blot, immunoprecipitation, radioimmunoassay, etc.

### Screening Assays

The subject cells are useful for in vitro assays and screening to detect agents that affect osteoblasts and cells in the osteogenic lineage. A wide variety of assays may be used for this purpose, including toxicology testing, immunoassays for protein binding; determination of cell growth, differentiation and functional activity; production of hormones; and the like.

In screening assays for biologically active agents, drugs, etc., the subject cells, usually a culture comprising the subject cells, is contacted with the agent of interest, and the effect of the agent assessed by monitoring output parameters, such as expression of markers, cell viability, and the like. The cells are typically in vitro cultured cells, and may include clonal cultures: e.g. split into independent cultures and grown under distinct conditions, for example with or without virus; in the presence or absence of other cytokines or combinations thereof. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc.

In addition to complex biological agents, candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists; etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 μl to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected parameters, or markers. For measuring the amount of a molecule that is present, a convenient method is to label a molecule with a detectable moiety, which may be fluorescent, luminescent, radioactive, enzymatically-active, etc., particularly a molecule specific for binding to the parameter with high affinity Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et el. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

### Kits

The culture systems of the invention are optionally packaged in a suitable container with written instructions for a desired purpose. Such formulations may comprise medium, growth factors, etc., in a form suitable for combining with cells prior to culture.

For further elaboration of general techniques useful in the practice of this invention, the practitioner can refer to standard textbooks and reviews in cell biology, tissue culture, and embryology. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Other features and advantages of the invention will be apparent from the description of the preferred embodiments, and from the claims. The following examples are offered by way of illustration and not by way of limitation.

### EXPERIMENTAL

### Material and Methods

Isolation and Culture of Mouse BMSCs. Isolation of MSCs from mouse bone marrow was according to Peister et al. (2004) Blood 103(5):1662-8. Female and male BALB/c mice 3 weeks old were individually euthanized using CO₂. The femurs and tibiae were removed, cleaned of all connective tissue, and placed on ice in 2 mL complete isolation media (CIM). CIM consisted of α-MEM (Invitrogen, Carlsbad, Calif.) supplemented with 20% fetal bovine serum (FBS; Atlanta Biologicals, Atlanta, Ga.), 100 U/mL penicillin (Invitrogen), 100 μg/mL streptomycin (Invitrogen), and 12 μM L-glutamine (Invitrogen). The ends of each tibia and femur were clipped to expose the marrow, flushed out using a 20 gauge needle, and centrifuged for 1 minute at 1200 rpm. The pellet was resuspended in 1 mL CIM with a micropipette. The cells from 2 mice were plated in 10 mL CIM in a 100 mm culture dish.

After 24 hours, nonadherent cells were removed by washing with phosphate-buffered saline (PBS), and 10 mL fresh CIM was added. The adherent cells (passage 0) were washed, and media changed with fresh CIM every 3 days for a period of 1 week. After 1 week, the cells were washed with PBS and detached by incubation in 1 mL 0.25% trypsin/1 mM ethylenediaminetetraacetic acid (EDTA; Invitrogen) for 2 minutes at 37° C. The cells that did not lift in 2 minutes were discarded. The trypsin was neutralized by the addition of 5 mL CIM, and all the cells (passage 1) from one dish were replated in 10 mL CIM in a 100 mm culture dish. The CIM was replaced every 3 days. After 1 week, the cells were lifted by incubating with trypsin/EDTA for 2 minutes at 37° C. The cells (passage 2) were then expanded by plating at 50 cells/cm² in complete expansion media (CEM) consisting of Iscove modified Dulbecco medium (IMDM; Invitrogen) supplemented with 9% FBS, 9% Horse serum (Invitrogen), 100 U/mL penicillin, 100 μg/mL streptomycin, and 12 μM L-glutamine. The CEM was replaced every 3 to 4 days. After 1 to 2 weeks, the cells (passage 3) were lifted by incubation with trypsin/EDTA for 2 minutes at 37° C. The passage 3 cells were either frozen or expanded further by plating at 50 cells/cm² and incubation in CEM.

Osteogenic and Adipogenic Differentiation of BMSCs. Osteogenic differentiation was induced by culturing cells in osteogenic medium (OS-medium, Cell Applications, Inc. CA) for 7 days. Adipogenic differentiation was induced by culturing cells in osteogenic medium (AD-medium, Cell Applications, Inc. CA) for 7 days.

After osteogenic induction cells were stained with alizarin red S (AR-S, Sigma, San Louis, Ind.). Cells were rinsed in PBS and incubated with 40 mM AR-S (pH 4.2) with rotation for 10 min, then rinsed 5 times with water followed by a 15 min wash with PBS with rotation to reduce nonspecific AR-S staining. The stained nodules were visualized using a light field microscope. Cells were washed with PBS and fixed with 10% formalin for 20 min. Cells were then washed twice with PBS and once with 60% isopropyl alcohol, and stained with Oil red O solution (Sigma). The stained nodules were observed through the microscope.

For detection of bone mineralization, we followed previously published methods were utilized. After culturing MSCs for 14 days, the media was aspirated and cells stained with alkaline phosphatase (StemTAG, Cell Biolabs, Inc. CA) according to the manufacturer's protocol and observed under light microscopy.

An alkaline phosphatase (ALP) assay was performed in cell layers by calorimetric assay of enzyme activity using an Alkaline phosphatase kit (Cell Biolab, Inc.), following the manufacturer' instructions. Cell layers were washed 3 times with PBS, then total proteins extracted using the Protein Extract Reagents kit (Pierce, Rockford, Ill.), followed by centrifugation to remove cellular debris. Fifty μL of lysate was then mixed with 50 μL of the freshly prepared calorimetric substrate para-nitrophenyl phosphate, and incubated at 37° C. for 30 min. The enzymatic reaction was stopped by adding 50 μL of 0.2 N NaOH. The optical density of the yellow product para-nitrophenol was determined by a HTS 7000 Plus Bio Assay reader (PE, USA) at 405 nm. Protein concentration of the cell lysates was measured with a BCA Protein Assay Kit (Pierce) and ALP activity was then expressed as O.D.405 nm/mg of protein.

Cytokine induction studies culture. At passage 5 or 6, MSC were placed in 60 mm culture dishes at a density of 2.5×10⁵ cells/well in complete expansion media (CEM). When cells reached 80-90% confluence, growth medium was supplemented with 10 ng/ml of TGF-β1 for 14 days. Media was changed every other day. Total RNA was isolated after 14 days using TRIzol (Invitrogen). RNA was dissolved in ddH₂O and stored at −80° C. The yield of RNA was determined by measuring absorbance at 260 nm.

Real-time RT-PCR. Reverse transcriptase (RT) reactions were annealed at 24° C. for 10 min, followed by first-strand cDNA synthesis at 48° C. for 1 hr and heat inactivation at 95° C. for 5 min. The resulting cDNA was stored at −20° C. until assayed by real-time PCR.

Real-time PCR analysis was performed using SYBR Green PCR core reagents (Applied Biosystems, Foster City, Calif.) following the manufacturer's protocol on an ABI Prism 7700 Sequence Detection System. All primers (see Table 1) were designed using the Primer3 program (Whitehead Institute, Cambridge, Mass.). Briefly, the real-time PCR reactions were performed with 10 μl each of SYBR Green kit master mix, and forward and reverse primers. Six μL of cDNA sample was added to 54 μL of final reaction mixture. The 384-well real-time PCR format included six 2-fold dilutions in triplicate of the plasmid DNA standards. The wells of the plate were sealed with optical adhesive covers (Bio-Rad Laboratories, Hercules, Calif.) and centrifuged at low speed (300×g, 5 min) to ensure complete mixing. Each sample was analyzed at least in triplicate.

Standard PCR involved activation of DNA polymerase followed by 40 cycles of denaturation at 94° C. for 30 s, annealing at 60° C. for 30 s, and extension at 72° C. for 30 s. The PCR threshold cycle number (C_{T}) for each sample was calculated at the point where the fluorescence exceeded the threshold limit. The threshold limit was fixed along the linear logarithmic phase of the fluorescence curves at 10 to 20 standard deviations (SDs) above the average background fluorescence. Relative expression levels were calculated using the standard curve method recommended by Applied Biosystems.

Statistical analysis. We performed three or more independent sets of the experiments, and each experiment was run at least three times. Data were shown as means ±SD and analyzed by a paired analysis of variance. P values were described in figures and P<0.05 was considered as statistically significant.

### Results

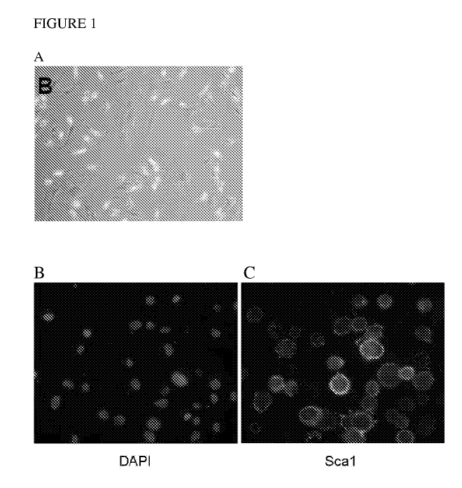

Bone Marrow-Derived MSC Isolation and Characterization. Marrow was isolated from the long bones of mice and seeded in culture dishes. Cells at passage 5-7 were used in the experiments. FIG. 1A shows the classical spindle morphology characteristic of BMSCs. Stem cell antigen-1 (Sca-1) is an 18-kDa mouse glycosyl phosphatidylinositol-anchored cell surface protein and has been used routinely in combination with negative selection against mature markers for enrichment of stem and progenitor cells (Holmes and Stanford (2007). Stem Cells 25(6):1339-47). In order to confirm if Sca-1 was expressed on BMSCs, immunocytochemistry was performed. FIG. 1C demonstrates that almost all BMSCs were found to express Sca-1 at the cell surface. In contrast, no positive staining was observed with BMSCs exposed to an isotype control IgG (FIG. 1B).

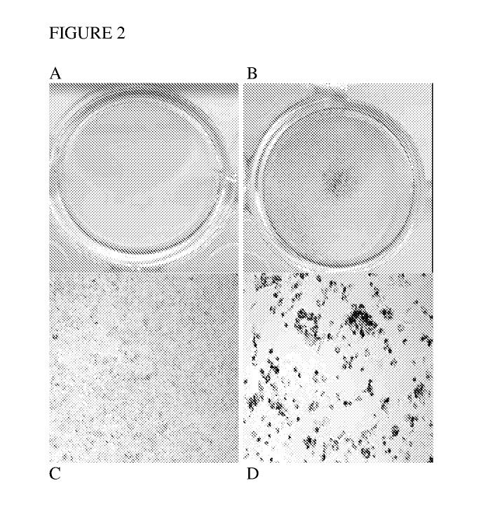

In order to identify if multipotency was maintained, BMSCs were cultured in osteogenic or adipogenic induction medium for 7 days. Cells were then washed with PBS and stained with alizarin red or red oil O. FIG. 2A reveals ˜that 80% of cells in the osteogenic medium were positive for alizarin red staining indicating osteogenic differentiation, whereas BMSCs which were cultured in IMDM medium were negative (FIG. 2B). Similarly, BMSCs cultured in adipogenic medium stained positively with red oil O indicating adipogenic differentiation (FIG. 2C). No staining was observed with BMSCs cultured in basic control medium (FIG. 2D).

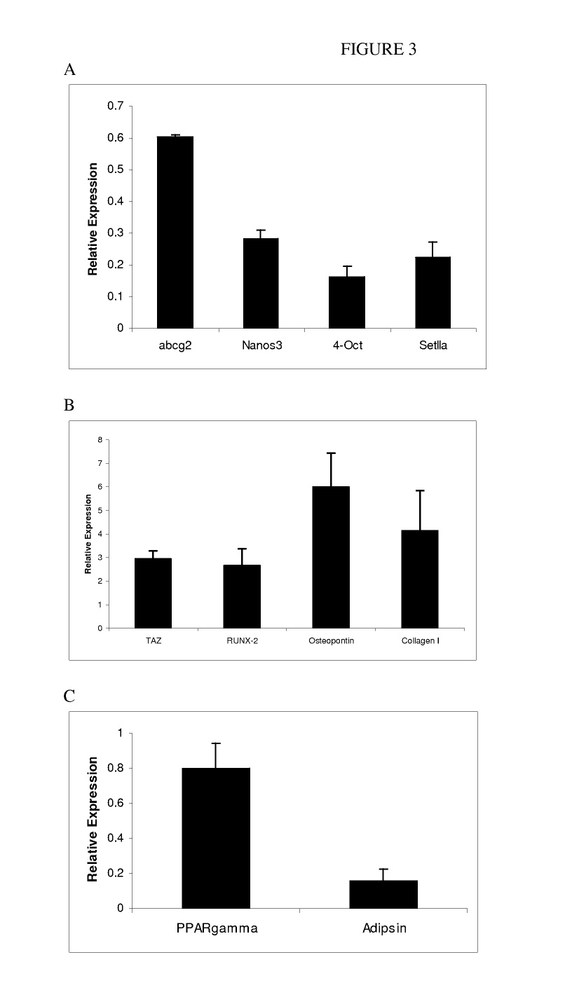

The effect of TGF-β1 on differentiation of BMSCs. TGF-β has been shown to stimulate chondrocytic differentiation in vitro, but whether it modulates osteoblastic differentiation remains unclear. In order to clarify this question, BMSCs were cultured in IMDM media with or without TGF-β1 for 14 days. mRNA was then isolated and RT-PCR was perfused to examine the expression of stem cell markers in BMSCs. FIG. 3 shows that TGF-β1 reduced expression of abcg2 (0.60±0.00), nanos3 (0.28±0.03), Oct4 (0.16±0.03), and stella (0.22±0.05), indicating downregulation of self-renewal pathways. FIG. 3 also shows that the expression of osteogenic transcript factors, TAZ and runx2, was increased 2.9±0.3 and 2.6±0.7 fold, respectively, in the TGF-β1 treated group relative to untreated controls. Similarly, the expression of osteoblast markers, osteopontin and collagen I was enhanced 6.0±1.4 and 4.1±1.7 fold in the treated group (FIG. 3). In contrast, mRNA levels of adipocyte markers, PPARγ2 and adipsin, were reduced to 0.80±0.14 and 0.15±0.06, respectively, in the TGF-β1 treated group (FIG. 3). Surprisingly, mRNA levels of chondrocyte markers (collagen types II, IX and aggrecan) were reduced 50% relative to controls.

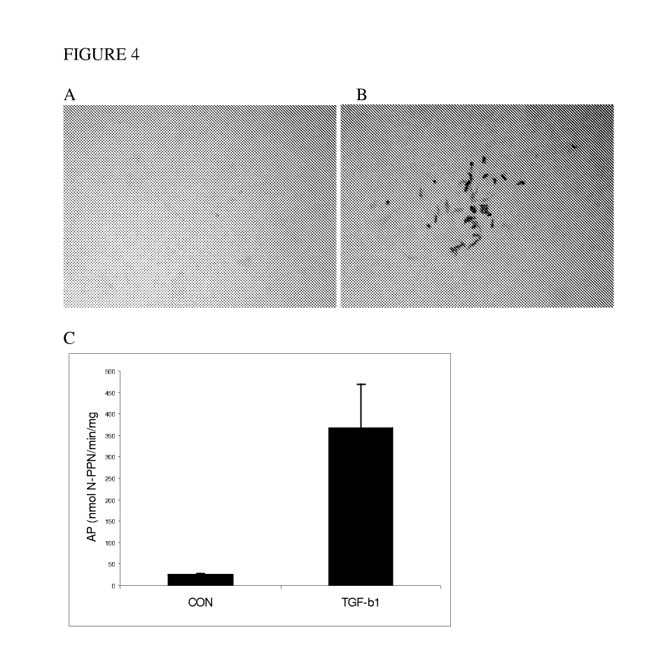

To further confirm these findings, the expression and activity of alkaline phosphatase (AP), a marker of bone differentiation, was investigated in TGF-β1 treated BMSCs. FIG. 4A shows that the AP staining increased significantly after days or weeks of TGF-β1 treatment compared to untreated cells. Furthermore, AP activity was dramatically increased to 368±100 nmol N—PPN/min/mg in TGF-β1 treated BMSCs, compared to 25±2 nmol N—PPN/min/mg in untreated cells (FIG. 4B).

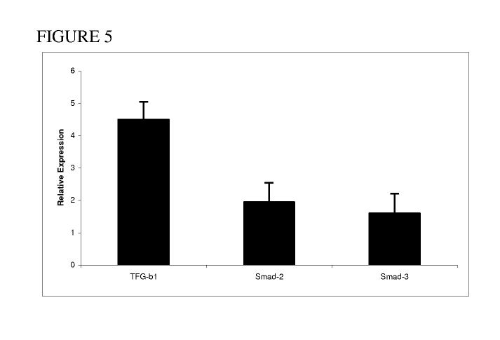

To better understand the effects of TGF-β1 on BMSC osteogenic differentiation, we examined for expression of downstream effectors of TGF-β1 signaling by RT-PCR. FIG. 5 shows that mRNA levels of smad 2 and smad 3 were increased 1.9±0.6 and 1.6±0.6 fold, respectively, after BMSCs were treated with TGF-β1. Furthermore, the expression of TGF-β1 was increased 4.5±0.6 fold.

To our knowledge, this is the first study to report on the osteogenic potential of recombinant TGF-β1 in bone marrow-derived MSCs in vitro. Our data confirm that TGF-β1 not only significantly enhances cell proliferation, but also stimulates osteogenesis in BMSCs as assessed by immunohistochemical, biochemical, and gene expression studies performed herein. Furthermore, this study also demonstrates the ability of TGF-β1 to significantly promote TAZ and runx2 mRNA levels in BMSCs in vitro

Our results indicate that a more physiological agonist such as TGF-β1 may possess therapeutic potential as an osteogenic induction factor, compared to the traditional induction protocols involving a combination of dexamethasone, β-glycerophosphate and ascorbic acid. Previous studies have shown that osteogenic factors such as BMP-2 and BMP-4 are absent during the whole healing process in humans (Zimmermann et al. (2005) Bone 36(5): 779-85), while TGF-β1 was shown to rise in peripheral blood dramatically at two weeks post-trauma and for at least an additional six months. Therefore, it is possible that TGF-β1 may play a role in bone fracture healing and that exogenous supplementation may overcome other physiological limitations such as enzymatic degradation or cross-talk from other signaling pathways induced post-injury.

Several studies have been shown that TGF-β1 stimulated chondrocytic differentiation in vitro (Han et al. (2005) J Cell Biochem 95(4):750-62; Mehlhorn et al. (2006) Tissue Eng 12(10):2853-62). However, in our system TGF-β1 decreased the expression of chondrogenic markers, such as collagen II, collagen IX and aggrecan at 14 days. The explanation may be that TGF-β1 acts sequentially to drive successive stages of differentiation during cartilage and bone development.

Large bodies of evidence indicate that Runx2 is an essential transcription factor for bone formation (Ducy et al. (2000) Science 289(5484):1501-4; Yamaguchi et al. (2000) Endocr Rev 21(4):393-411). However, it has been suggested that other transcriptional regulators are cooperatively involved in the osteogenic action of Runx-2 because the transcriptional activity of Runx2 itself is relatively weak Kanno e al. (1998) Mol Cell Biol 18(5):2444-54) TAZ, a transcriptional coactivator with PDZ-binding motif, interacts with a variety of transcription factors and exhibits transcriptional regulatory functions. TAZ is believed to regulate gene expression during embryogenesis and development of bone, muscle, fat, lung, heart, and limbs. Furthermore, a recent study indicates that TAZ acts as a transcriptional regulator for the differentiation of MSCs into osteoblast cells (Hong et al, supra). In this case, TAZ functions as a coactivator of Runx2 and acts as a corepressor of PPARγ, which is a master regulator of adipocyte differentiation. These reports reveal that TAZ plays an important role in MSC differentiation; however, the exact upstream triggers of this pathway were not elucidated. Our results showed that activation of TGF-β-Smad signaling promoted TAZ and Runx2 expression and osteoblast differentiation. Transcripts of several components of TGF-β1-Smad pathway were up-regulated during differentiation: TGF-β1, Samd-2, Samd-3, and Act-2a on day 14. This study is the first to reveal that TGF-β1 enhance the TAZ mRNA level mediated by the TGF-β-Smad signaling pathway.

In conclusion, the identification of functional roles of TGF-β1 in osteoblast differentiation provides a novel insight into understanding the molecular mechanism of the commitment of mesenchymal stem cells in bone marrow and may allow as to develop new therapeutic for bone disease such as osteoporosis, bone fracture.

TABLE 1
Primers used for real-time polymerase chain reaction

| | Sequence | |
|---|---|---|
| Gene marker | identifier | Primers sequence |
| Runx2 | SEQ ID NO:1 | (5′-3′) CCC AGC CAC CTT TAC CTA CA |
| | SEQ ID NO:2 | (3′-5′) TAT GGA GTG CTG CTG GTC TG |
| TAZ | SEQ ID NO:3 | (5′-3′) TCC CCA CAA CTC CAG AAG AC |
| | SEQ ID NO:4 | (3′-5′) CAA AGT CCC GAG GTC AAC AT |
| Osteopontin | SEQ ID NO:5 | (5′-3′) CAC TCC AAT CGT CCC TAC |
| | SEQ ID NO:6 | (5′-3′) AGA CTC ACC GCT CTT CAT |
| Osteocalcin | SEQ ID NO:7 | (5′-3′) AAG CCC AGC GAC TCT GAG TC |
| | SEQ ID NO:8 | (3′-5′) GCT CCA AGT CCA TTG TTG AGG |
| Collagen I | SEQ ID NO:9 | (5′-3′) ATG GAG ACA GGT CAG ACC TGT GT |
| | SEQ ID NO:10 | (3′-5′) TCG GTC ATG CTC TCT CCA AAC |
| Adipsin | SEQ ID NO:11 | (5′-3′) GCT ATC CCA GAA TGC CTC GTT |
| | SEQ ID NO:12 | (3′-5′) GCG TGC CGG GTT CCA |
| PPARgamma | SEQ ID NO:13 | (5′-3′) AAC CAT TGG GTC AGC TCT TG |
| | SEQ ID NO:14 | (5′-3′) GAT GGA AGA CCA CTC GCA TT |
| TGF-β1 | SEQ ID NO:15 | (5′-3′) AAC AAT TCC TGG CGT TAC CTT |
| | SEQ ID NO:16 | (3′-5′) ATT CCG TCT CCT TGG TTC AG |
| Smad-2 | SEQ ID NO:17 | (5′-3′) GGA AAG GGT TGC CAC ATG TTA T |
| | SEQ ID NO:18 | (3′-5′) GCA GTT TTC GAT TGC CTT GAG |
| Smad-3 | SEQ ID NO:19 | (5′-3′) GGG CCT ACT GTC CAA TGT CAA |
| | SEQ ID NO:20 | (3′-5′) CGC ACA CCT CTC CCA ATG T |
| Smad-4 | SEQ ID NO:21 | (5′-3′) CGC TTT TGC TTG GGT CAA CT |
| | SEQ ID NO:22 | (5′-3′) TGT GCA ACC TCG CTC TCT CA |

What is claimed is:

1. A method for obtaining osteogenic progenitor cells, the method comprising:
culturing isolated mesenchymal stem cells in vitro in the presence of a TGF-β factor at a concentration of from about 100 pg/ml to about 100 ng/ml for a period of time sufficient to induce osteogenic differentiation.

2. The method according to claim 1, wherein the TGF-β factor is present at around about 10 ng/ml.

3. The method of claim 1, wherein the TGF-β factor is TGF-β1.

4. The method of claim 1, wherein the TGF-β factor is TGF-β3.

5. The method of claim 1, further comprising the step of isolating osteogenic progenitor cells from the culture.

6. The method of claim 1, wherein TAZ activity is upregulated.

7. The method according to claim 1, wherein the mesenchymal stem cells are isolated from adipose tissue.

8. The method according to claim 1, wherein the mesenchymal stem cells are isolated from bone marrow.


SEQUENCE LISTING (22 sequences)

SEQ ID NO: 1 - DNA, 20 bp, Artificial Sequence
  oligonucleotide
cccagccacctttacctaca

SEQ ID NO: 2 - DNA, 20 bp, Artificial Sequence
  oligonucleotide
tatggagtgctgctggtctg

SEQ ID NO: 3 - DNA, 20 bp, Artificial Sequence
  oligonucleotide
tccccacaactccagaagac

SEQ ID NO: 4 - DNA, 20 bp, Artificial Sequence
  oligonucleotide
caaagtcccgaggtcaacat

SEQ ID NO: 5 - DNA, 18 bp, Artificial Sequence
  oligonucleotide
cactccaatcgtccctac

SEQ ID NO: 6 - DNA, 18 bp, Artificial Sequence
  oligonucleotide
agactcaccgctcttcat

SEQ ID NO: 7 - DNA, 20 bp, Artificial Sequence
  oligonucleotide
aagcccagcgactctgagtc

SEQ ID NO: 8 - DNA, 21 bp, Artificial Sequence
  oligonucleotide
gctccaagtccattgttgagg

SEQ ID NO: 9 - DNA, 23 bp, Artificial Sequence
  oligonucleotide
atggagacaggtcagacctgtgt

SEQ ID NO: 10 - DNA, 21 bp, Artificial Sequence
  oligonucleotide
tcggtcatgctctctccaaac

SEQ ID NO: 11 - DNA, 21 bp, Artificial Sequence
  oligonucleotide
gctatcccagaatgcctcgtt

SEQ ID NO: 12 - DNA, 15 bp, Artificial Sequence
  oligonucleotide
gcgtgccgggttcca

SEQ ID NO: 13 - DNA, 20 bp, Artificial Sequence
  oligonucleotide
aaccattgggtcagctcttg

SEQ ID NO: 14 - DNA, 20 bp, Artificial Sequence
  oligonucleotide
gatggaagaccactcgcatt

SEQ ID NO: 15 - DNA, 21 bp, Artificial Sequence
  oligonucleotide
aacaattcctggcgttacctt

SEQ ID NO: 16 - DNA, 20 bp, Artificial Sequence
  oligonucleotide
attccgtctccttggttcag

SEQ ID NO: 17 - DNA, 22 bp, Artificial Sequence
  oligonucleotide
ggaaagggttgccacatgttat

SEQ ID NO: 18 - DNA, 21 bp, Artificial Sequence
  oligonucleotide
gcagttttcgattgccttgag

SEQ ID NO: 19 - DNA, 21 bp, Artificial Sequence
  oligonucleotide
gggcctactgtccaatgtcaa

SEQ ID NO: 20 - DNA, 19 bp, Artificial Sequence
  oligonucleotide
cgcacacctctcccaatgt

SEQ ID NO: 21 - DNA, 20 bp, Artificial Sequence
  oligonucleotide
cgcttttgcttgggtcaact

SEQ ID NO: 22 - DNA, 20 bp, Artificial Sequence
  oligonucleotide
tgtgcaacctcgctctctca